(19) United States
(12) Patent Application Publication
(10) Pub. No.: US 2022/0183691 A1
(43) Pub. Date: Jun. 16, 2022

(54) MEDICAL DEVICE

(71) Applicant: OLYMPUS CORPORATION, Tokyo (JP)

(72) Inventor: Toshihiro YOSHII, Hachioji-shi (JP)

(73) Assignee: OLYMPUS CORPORATION, Tokyo (JP)

(21) Appl. No.: 17/683,437

(22) Filed: Mar. 1, 2022

Related U.S. Application Data
(63) Continuation of application No. PCT/JP2019/042971, filed on Nov. 1, 2019.

(51) Int. Cl. A61B 17/122; A61B 17/128

(52) U.S. Cl. CPC A61B 17/122; A61B 17/1285

(57) ABSTRACT

A clip device includes a clip unit including an openable arm; a hook detachably connected to the clip unit; a wire configured to operate the clip unit and connected to the hook, a tubular member through which the hook is insertable; and a claw portion provided in the hook. In a first state in which the hook is connected with the clip unit, the claw portion is configured to have a radial dimension larger than an inner diameter of the tubular member, and in a second state in which the hook is disconnected from the clip unit, the radial dimension of the claw portion is equal to or less than the inner diameter of the tubular member.

This application is a continuation application of PCT International Application No. PCT/JP2019/042971, filed Jan. 11, 2019. The entire disclosure of the PCT International Application is hereby incorporated by reference herein.

### TECHNICAL FIELD

The present disclosure relates to a medical device, more specifically, relates to a medical device including a clip unit for ligating the tissues.

### BACKGROUND ART

Ligation using a clip unit is known as a procedure performed using an endoscope. The clip unit includes a pair of arms. When the pair of arms are pulled by a predetermined amount while the pair of arms clamp the tissue, the pair of arms are locked with the tissue strongly tightened.

The clip unit is introduced into the body while being attached to an applicator. Since the clip unit is indwelled in the body with the tissue ligated, it is necessary for the pair of arms to be separated from the applicator after the pair of arms are locked.

There are several known aspects for releasing the connection between the applicator and the clip unit. In the configuration described in Published Japanese Translation No. 2008-526376 of the PCT International Publication, the applicator and the clip unit are connected by the clip of the applicator grasping the proximal end portion of the clip unit. When the clip of the applicator is retracted to move into the expansion chamber, the clip opens and the connection between the applicator and the clip unit is released.

The operator closes the pair of arms to tighten the target tissue, and then confirms that the ligation is sufficient by checking the grasping position. In a case in which it is determined that the ligation is insufficient, the pair of arms are opened to release the tightening with respect to the target tissue, and the ligation operation is performed again. Then, when it is determined that the ligation is sufficient, the pair of arms are locked. At this time, since the operation force (locking force) required to lock the pair of arms of the clip unit is significantly larger than the force required to ligate the target tissue, the operator may recognize the gap in the force and perform the procedures without any mistake of accidentally locking the pair of arms.

The amount of operation force (locking force) required to lock the pair of arms of the clip unit varies depending on the reaction force received from the target tissue clamped by the pair of arms. The locking force increases once the target tissue is more rigid.

In the case of applying the method of releasing the connection by the deformation to the clip unit, if the connection is released before the locking operation is finished, it is difficult to ligate the tissue. Accordingly, the operation force (releasing force) required to release the connection is generally set to be larger than the locking force. In the view point of securing the force gap described above, there is a case in which the locking force is set to be further larger. Here, if the releasing force is set based on the locking force when the arm clamps the tissue with the average rigidness, it is possible that in a case of grasping the rigid tissue, the connection may be released even though the locking operation is not completed. It is possible to avoid such a situation by setting the releasing force based on the locking force when the arms clamp the rigid tissue; however, the releasing force becomes significantly large to make the operation to be difficult.

### SUMMARY

According to an aspect of the present disclosure, a clip device includes a clip unit having an openable arm; a hook detachably connected to the clip unit; a wire configured to operate the clip unit and connected to the hook, a tubular member through which the hook is insertable; and a claw portion provided in the hook, wherein in a first state in which the hook is connected with the clip unit, the claw portion is configured to have a radial dimension larger than an inner diameter of the tubular member, and in a second state in which the hook is disconnected from the clip unit, the radial dimension of the claw portion is equal to or less than the inner diameter of the tubular member.

According to another aspect of the present disclosure, a clip device includes a clip unit having an arm; a hook detachably connected to the clip unit; a wire configured to operate the clip unit and connected to the hook; and a pair of claws provided in the hook, wherein the pair of claws are disposed at positions sandwiching the clip unit in a first direction orthogonal to a longitudinal direction of the hook, and the pair of claws are disposed at asymmetrical positions in a second direction orthogonal to the first direction and the longitudinal direction.

According to a further aspect of the present disclosure, a tissue closure method includes pulling a wire to grasp a tissue by a clip detachably connected to the wire; causing a link member to come into contact with an inner circumferential surface of a tubular member by pulling the wire in a state in which the tissue is grasped by the clip, the link member being configured to detachably connect the clip and the wire, and the wire being insertable through the tubular member; releasing a connection between the link member and the clip by pulling the wire in a state in which the link member is in contact with the inner circumferential surface of the tubular member; and separating the link member from the tubular member by pulling the wire in a state in which the connection between the link member and the clip is released.

### BRIEF DESCRIPTION OF DRAWINGS

FIG. 1 is a view showing an overall configuration of a ligation device according to a first embodiment of the present disclosure.

FIG. 2 is a view showing a clip unit of the ligation device.

FIG. 3 is a cross-sectional view showing the clip unit.

FIG. 4 is a cross-sectional view showing the clip unit from a different direction from that in FIG. 3.

FIG. 5 is an enlarged cross-sectional view showing a clip attachment portion in the ligation device.

FIG. 6 is an enlarged view of the hook.

FIG. 7 is a cross-sectional view taken along the line I-I in FIG. 6.

FIG. 8 is a cross-sectional view taken along the line II-II in FIG. 6.

FIG. 9 is a schematic view showing a process of the operation to release the connection between the arm and the hook in the ligation device.

FIG. 10 is a schematic view showing a process of the operation to release the connection between the arm and the hook.

FIG. 11 is a view showing a state when a claw portion of the hook is close in a second state.

FIG. 12 is a schematic view showing a process of the operation to release the connection between the arm and the hook.

FIG. 13 is a schematic view showing a process of the operation to release the connection between the arm and the hook.

FIG. 14 is a graph showing a relationship between a traction amount of an operation wire and the force in the operation to release the connection.

FIG. 15A is a cross-sectional view showing a hook based on a modification example in a first state.

FIG. 15B is a cross-sectional view showing the hook based on the same modification example in the second state.

### DESCRIPTION OF EMBODIMENTS

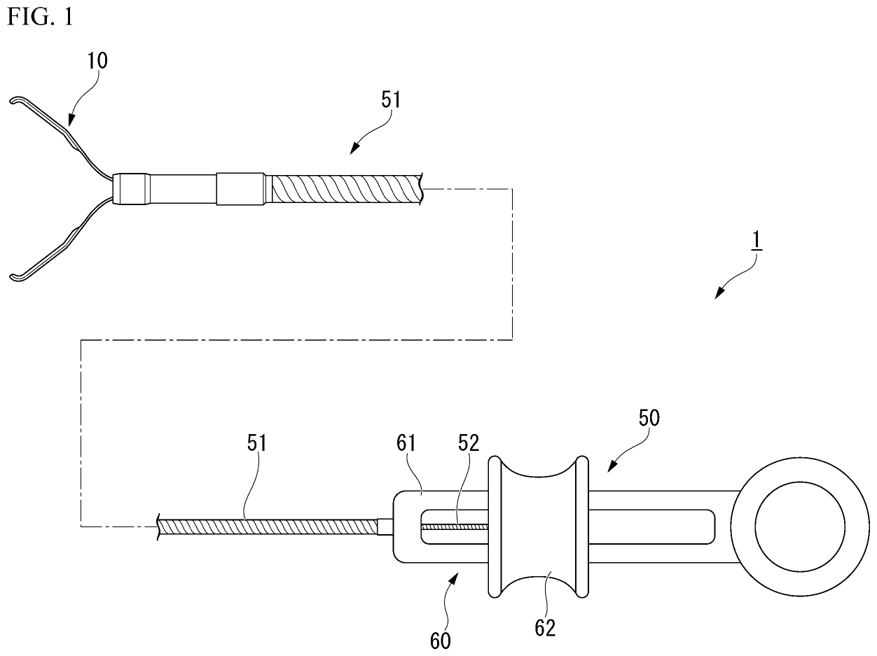

An embodiment of the present disclosure will be described with reference to FIG. 1 to FIG. 14.

FIG. 1 is a view showing an appearance of the ligation device 1 as a medical device according to the present embodiment. The ligation device 1 includes a clip unit 10 that is indwelled in the body and an applicator 50 configured to operate the clip unit 10. The clip unit 10 is attached to a distal (tip end) of the applicator 50.

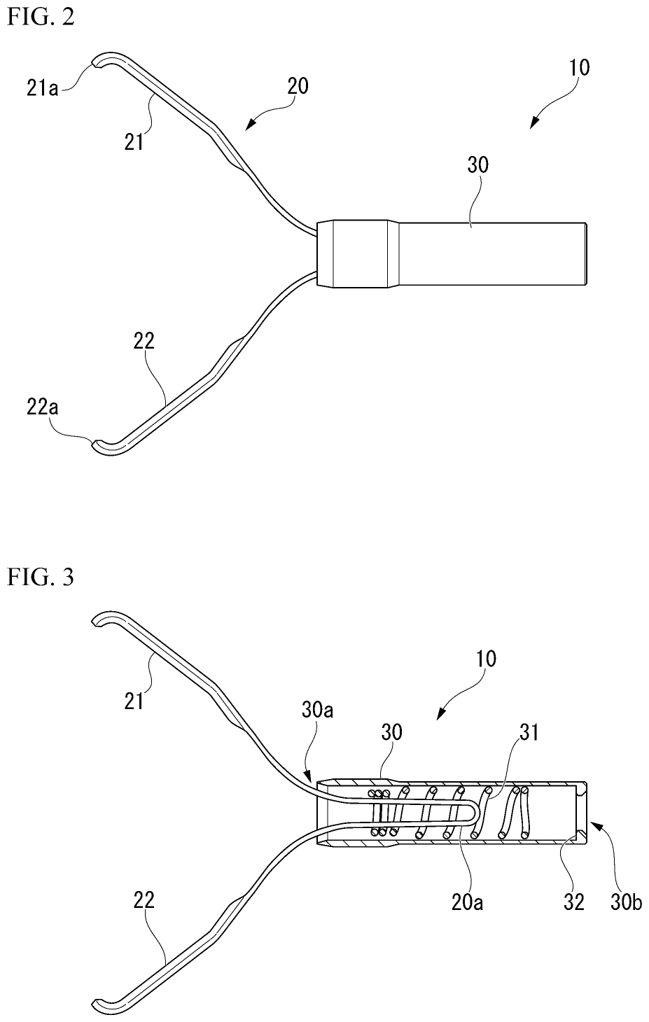

FIG. 2 is a view showing an appearance of the clip unit 10. FIG. 3 is a cross-sectional view of the clip unit 10. As shown in FIG. 2, the clip unit 10 includes an arm portion 20 and a pressing tube 30 in which a part of the arm portion 20 is accommodated.

The arm portion 20 includes a first arm 21 and a second arm 22 as a pair of arms. The first arm 21 and the second arm 22 have claws 21a and 22a at a distal end portion thereof, respectively. As shown in FIG. 3, the first arm 21 and the second arm 22 are connected at the proximal end portion 20a of the arm portion 20. The proximal end portion 20a is formed in a U shape.

The arm portion 20 is made of a metal containing an alloy. Examples of the material of the arm portion 20 include the stainless steel, the cobalt-chromium alloy, and the nickel-titanium alloy.

The first arm 21 and the second arm 22 are expanded in an initial state shown in FIG. 2. When the first arm 21 and the second arm 22 approach each other from the initial state, a biasing force for returning to the initial state is generated due to the elastic force of the material.

The pressing tube 30 is a tubular member made of the metal, the resin, or the like. As shown in FIG. 3, the proximal end portion 20a of the arm portion 20 is accommodated in the pressing tube 30. The distal end of the arm portion 20 is projecting from the distal end opening 30a of the pressing tube 30. The proximal end opening 30b of the pressing tube 30 is smaller than the distal end opening 30a.

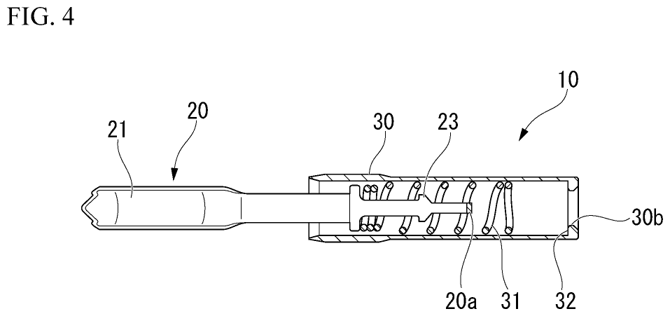

FIG. 4 is a view showing the inside of the pressing tube 30 as viewed from a direction different from that in FIG. 3. As shown in FIG. 4, a locking portion 23 is provided in an intermediate portion of each arm of the arm portion 20, and the dimensions of the respective arms 21, 22 are increased in the locking portion 23 (only the first arm 21 is visible in FIG. 4). Each locking portion 23 can pass through the proximal end opening 30b when the first arm 21 and the second arm 22 approach each other. When the first arm 21 and the second arm 22 are separated from each other after passing through the proximal end opening 30b, the locking portion 23 cannot pass through the proximal end opening 30b. As a result, the arm portion 20 is locked with the pair of arms closed.

A coil spring 31 is arranged inside the pressing tube 30. The front end of the coil spring 31 can come into contact with the rear surfaces of the first arm 21 and the second arm 22. The rear end of the coil spring 31 can come into contact with the rear end surface 32 of the pressing tube having the proximal end opening 30b.

The basic structures of the arm portion 20 and the pressing tube 30 described above are well-known and are disclosed, for example, in PCT International Publication No. 2014/181676.

As shown in FIG. 1, the applicator 50 includes an elongated insertion portion 51, an operation wire (wire) 52 passed through the insertion portion 51, and an operation portion 60 connected to the insertion portion 51.

As the insertion portion 51, for example, a sheath formed of a coil can be adopted.

The operation unit 60 has a main body 61 connected to the insertion unit 51 and a slider 62 slidably attached to the main body 61.

As the operation wire 52, for example, a stranded wire made of a metal wire can be adopted. The proximal end portion of the operation wire 52 is connected to the slider 62. When the slider 62 is moved with respect to the main body 61, the operation wire 52 can be advanced and retracted in the insertion portion 51.

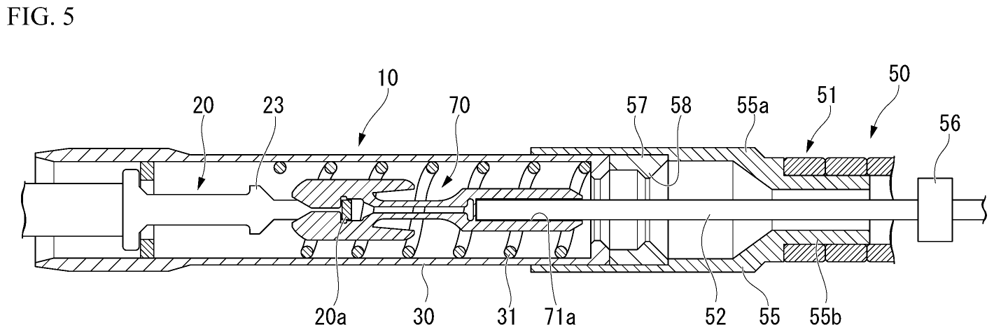

FIG. 5 is an enlarged cross-sectional view of the distal end portion of the applicator 50 to which the clip unit 10 is mounted.

A hook 70 that engages with the clip unit 10 is fixed to the distal end of the operation wire 52. As shown in FIG. 5, the distal end portion of the operation wire 52 enters the pressing tube 30, and the hook 70 and the proximal end portion 20a of the arm portion 20 are engaged with each other. The external dimensions of the hook 70 are slightly smaller than the inner diameter of the coil spring 31, and the hook 70 can pass through the coil spring 31 without interfering with the coil spring 31.

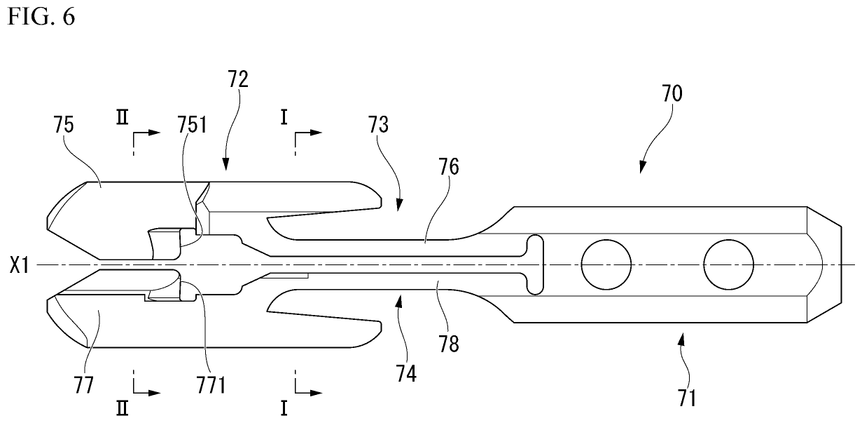

FIG. 6 is an enlarged view of the hook 70. The hook 70 has a rear portion 71 connected to the operation wire 52 and a front portion 72 that engages with the proximal end portion 20a.

The rear portion 71 is formed in a spindle shape that gradually shrinks as approaching both ends in the front-rear direction, and the rear portion 71 has a bottomed hole 71a (see FIG. 5) that opens at the rear end. The distal end of the operation wire 52 is entering the hole 71a. The operation wire 52 and the hook 70 are connected by, for example, the brazing or the like. In the present embodiment, the hook 70 and the operation wire 52 are connected while maintaining a coaxial state.

The front portion 72 has a pair of engaging arms 73, 74. The engaging arms 73, 74 clamp the arm portion 20 from a direction orthogonal to the open-close direction of the arm portion 20. The engaging arm 73 has a claw portion 75 that comes into contact with the arm portion 20, and a plate-shaped portion 76 that connects the claw portion 75 and the rear portion 71. Similar to the engaging arm 73, the engaging arm 74 has a claw portion 77 and a plate-shaped portion 78, and the engaging arm 74 has the same shape as that of the engaging arm 73. The engaging arms 73, 74 are provided at positions symmetrical with respect to the central axis X1 of the hook 70.

In a state in which the arm portion 20 and the hook 70 are connected with each other (first state), the claw portion 75 and the claw portion 77 are separated from each other, and the claw portion 75 and the claw portion 77 are in a positional relationship to be able to approach each other without the arm portion 20.

The claw portion 75 has an engaging surface 751 that comes into contact with the proximal end portion 20a of the arm portion 20. The engaging surface 751 is a flat surface extending in a direction perpendicular to the central axis X1. The claw portion 77 also has an engaging surface 771 similar to the engaging surface 751.

In the state shown in FIG. 5 in which the claw portion 75 and the claw portion 77 clamp and hold the arm portion 20, the outer shape of the front portion 72 configured by the claw portion 75 and the claw portion 77 is larger than that of the rear portion 71.

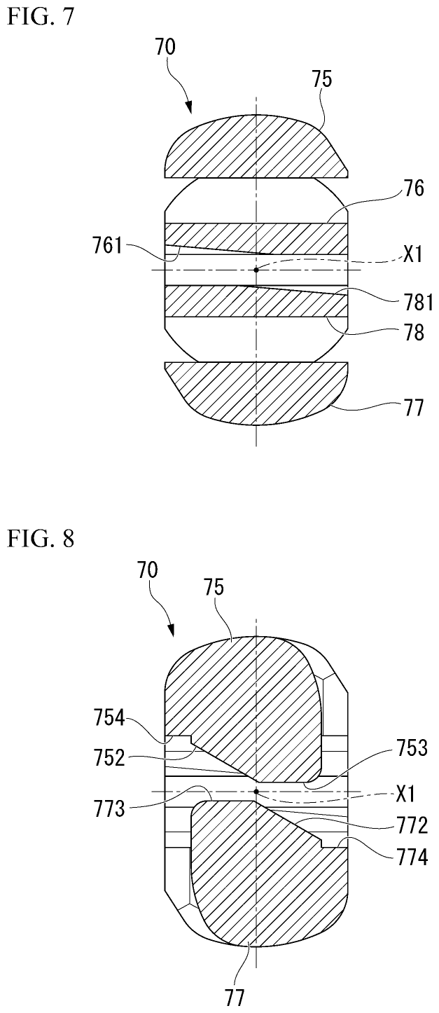

FIG. 7 is a cross-sectional view taken along the line I-I in FIG. 6 as a view seen from the front side of the hook 70. The plate-shaped portions 76, 78 are configured to have a generally rectangular plate-shaped cross section; however, the distal end portion connected to the claw portion is chamfered, and inclined surfaces 761, 781 are formed on the surface close to the central axis X1, respectively. The inclined surface 761 extends upward from the center in the width direction of the plate-shaped portion 76. The inclined surface 781 extends downward from the center of the plate-shaped portion 78 in the width direction. That is, the inclined surfaces 761, 781 are separated from the central axis X1 as they approach the widthwise end portion of the plate-shaped portion.

In the width direction of the plate-shaped portions 76, 78, the starting points of the inclined surfaces 761, 781 and the central axis X1 are substantially at the same position.

FIG. 8 is a cross-sectional view taken along the line II-II of FIG. 6. In the claw portions 75, 77, the surfaces facing each other (opposing surfaces) on the distal end side of the engaging surface are chamfered, and inclined surfaces 752, 772 are formed, respectively. The inclined surface 752 extends upward from the center of the claw portion 75 in the width direction. The inclined surface 772 extends downward from the center of the claw portion 77 in the width direction. The inclined surfaces 752, 772 are inclined in the same direction as that of the inclined surfaces 761, 781, and the inclined surfaces 752, 772 move away from the central axis X1 as they approach the widthwise end of the claw portion. In the opposing surfaces, the portions where the inclined surfaces 752, 772 are not formed are parallel portions 753, 773 extending in the left-right direction and parallel to each other. Receiving surfaces 754, 774 being parallel to the parallel portions 753, 773 extend from the end points of the inclined surfaces 752, 772.

The right side of the front portion of the claw portion 75 without the inclined surface 752 is smaller in the front view, and the left side of the front portion of the claw portion 77 without the inclined surface 772 is smaller in the front view.

As shown in FIG. 6, the rear end portions of the claw portions 75, 77 have a curved outer peripheral surface, and the external dimensions decrease as approaching the proximal end portions.

As shown in FIG. 5, a rigid guide pipe 55 is attached to the distal end of the insertion portion 51. The inner diameter of region at the distal end side of the guide pipe 55 is larger than the outer diameter of the pressing tube 30, and the pressing tube 30 can enter the guide pipe 55. The guide pipe 55 has a reduced-diameter portion 55a wherein the inner diameter thereof gradually decreases as approaching the proximal end, and the inner diameter of a small-diameter portion 55b on the proximal end side of the reduced-diameter portion 55a is the same as the minimum inner diameter of the reduced-diameter portion 55a. The outer dimension of the rear portion 71 of the hook 70 is smaller than the inner diameter of the small-diameter portion 55b, and the maximum outer dimension of the front portion 72 defined by the claw portions 75, 77 is larger than the inner diameter of the small-diameter portion 55b.

A stopper 56 is attached to the operation wire 52. Since the shape and dimensions of the stopper 56 are set such that the stopper 56 cannot enter the guide pipe 55, when the stopper 56 comes into contact with the rear end of the guide pipe 55, the operation wire 52 cannot move forward any further.

The operation of the ligation device 1 configured as described above when used will be described. The ligation device 1 is introduced into the body via the channel of the endoscopic. When inserting the ligation device 1 into the endoscope, the user retracts the slider 62 by a predetermined amount and inserts the ligating device 1 in a closed and unlocked state. The clip unit 10 in which the arm portion 20 is closed and the distal end portion of the insertion portion 51 may be inserted into the endoscope in a state of being accommodated in a separately prepared outer sheath.

When the ligation device 1 is projected from the channel opening at the distal end portion of the endoscope and then reduce the pulling force of the slider or retract the outer sheath, the arm portion 20 advances with respect to the pressing tube 30 due to its own elastic restoration force and the elastic restoration of the coil spring 31. As a result, the pair of arms 21, 22 are in an open configuration. When the stopper 56 comes into contact with the rear end of the guide pipe 55, the arm portion 20 cannot advance with respect to the pressing tube 30 such that the arm portion 20 is kept in the open configuration and the arm portion 20 does not slip off from the pressing tube 30.

When the user retracts the slider 62 with respect to the main body 61, the operation wire 52 is pulled and the arm portion 20 retracts with respect to the pressing tube 30. As a result, the pair of arms 21, 22 are closed to enter a closed configuration. The user can ligate the tissue by locating the tissue between the pair of arms 21, 22 and closing the pair of arms 21, 22. By advancing the slider 62 with respect to the main body 61 until the locking operation described later is performed, the pair of arms 21, 22 can be transitioned from the closed configuration to the open configuration again. Therefore, in the ligation device 1, the clip unit 10 can be operated by the operation wire 52 to re-grasp the tissue until the locking operation is performed.

As shown in FIG. 5, a regulation member 57 for preventing unintentional release of the connection between the proximal end portion 20a and the hook 70 is arranged in the guide pipe 55. The regulation member 57 has a regulation portion 58 having an inner diameter slightly larger than the outer diameter of the hook 70. In order for releasing the connection between the proximal end portion 20a and the hook 70, it is necessary for the engaging arms 73, 74 to be separated by a certain distance or more, however, there is not such a space in the regulation portion 58 for the engaging arm 73 and the engaging arm 74 to be sufficiently separated from each other. As a result, the engagement between the hook 70 and the proximal end portion 20a is not released until the engaging arms 73, 74 pass through the regulation portion 58, and the engagement state is suitably maintained.

When it is determined that the tissue located between the pair of arms 21, 22 may be ligated, the user performs the locking operation for fixing the arm portion 20 in the closed configuration. During the locking operation, the user further retracts the slider 62 with respect to the main body 61 beyond the range in which it is possible to perform the re-grasp. When the slider 62 is retracted, the operation wire 52 is pulled, and the pair of arms 21, 22 enter the pressing tube 30 in substantially parallel with each other while clamping the tissue. Furthermore, the locking portions 23 provided on the pair of arms 21, 22 approach each other and have a positional relationship in which they can pass through the proximal end opening 30b of the pressing tube 30.

The pair of locking portions 23 that have passed through the proximal end opening 30b and moved out of the pressing tube 30 are separated again when the force received from the operation wire 52 weakens, and the pair of locking portions 23 are in a positional relationship so as to not be able to pass through the proximal end opening 30b. As a result, the pair of locking portions 23 come into contact with the edge of the proximal end opening 30b to prevent the arm portion 20 from protruding from the pressing tube 30, and the arm portion 20 is locked to maintain the closed configuration.

During the process of the locking operation, the proximal end portion 20a and the hook 70 move out of the pressing tube 30 through the proximal end opening 30b; however, the engagement state between the proximal end portion 20a and the hook 70 is suitably maintained by the regulation member 57.

When the user further retracts the slider 62 after the arm portion 20 is locked, the engagement between the proximal end portion 20a and the hook 70 is released, and the clip unit 10 is separated from the applicator 50. Hereinafter, the operation of the hook 70 at the time of releasing the connection will be described in detail with reference to a schematic view.

When the slider 62 is retracted after the arm portion 20 is locked, the operation wire 52 is pulled. As a result, as shown in FIG. 9, the rear end portions of the claws 75, 77 come into contact with the inner surface of the reduced-diameter portion 55a. When the operation wire 52 is pulled, the rear end portions of the claws 75, 77 move along the inner surface of the reduced-diameter portion 55a and then enter the small-diameter portion 55b.

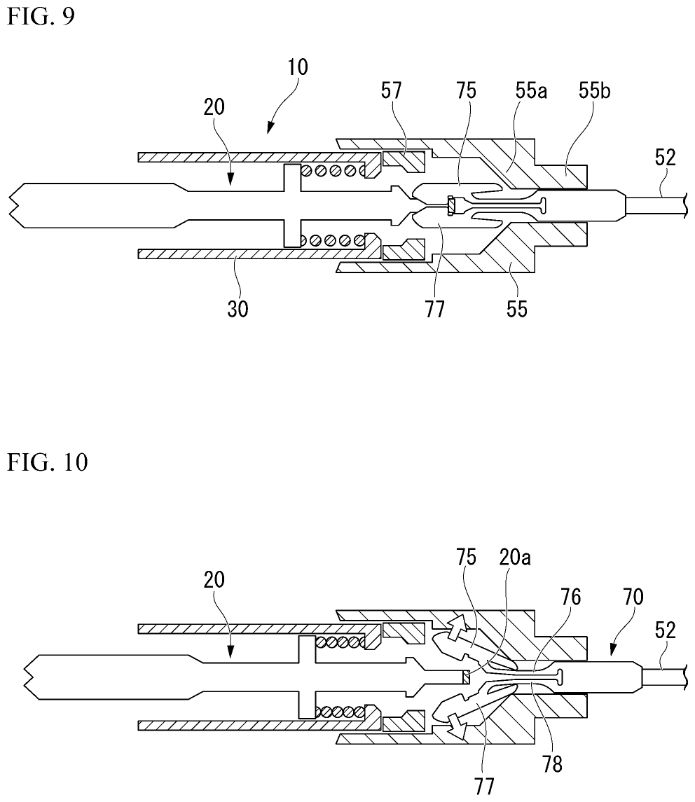

As the rear side of the claw portion enters the small-diameter portion 55b, the claw portions 75, 77 rotate about the connection portion with the plate-shaped portion as the rotation center, respectively. As a result, as shown in FIG. 10, the distal end portions of the claws 75, 77 move in a direction apart away from each other and separate from the proximal end 20a of the arm portion 20 . In this manner, the connection between the hook 70 and the arm portion 20 is released, and the pair of claw portions 75, 77 transitions to the second state in which the arm portion 20 does not exist therebetween.

When the operation wire 52 is further pulled after the connection is released, the distal end portions of the plate-shaped portion 76 and the plate-shaped portion 78 move in a direction approaching each other. The plate-shaped portions 76, 78 that approach each to be in close contact with each other cause the inclined surfaces 761, 781 into contact with each other, and start moving along the opposite inclined surfaces.

Together with the movement of the plate-shaped portions 76, 78, the claw portions 75, 77 also move. The opposite surfaces of the claws 75, 77 approach each other while being displaced in the left-right direction when viewed in the front view. The left-right direction in which the claw portions 75, 77 are displaced intersects the open-close direction in which the claw portions 75, 77 clamp the arm portion 20. The opposite surface of the claw portion 75 is displaced to the right side as the plate-shaped portion 76 moves along the inclined surface 781. The opposite surface of the claw portion 77 is displaced to the left side as the plate-shaped portion 78 moves along the inclined surface 761.

When the claws 75, 77 approach each other while being displaced in the left-right direction, the inclined surface 752 and the inclined surface 772 come into contact with each other. Thereafter, the claw portions 75, 77 move along the opposite inclined surfaces, and stop the relative movement when a balanced state is reached.

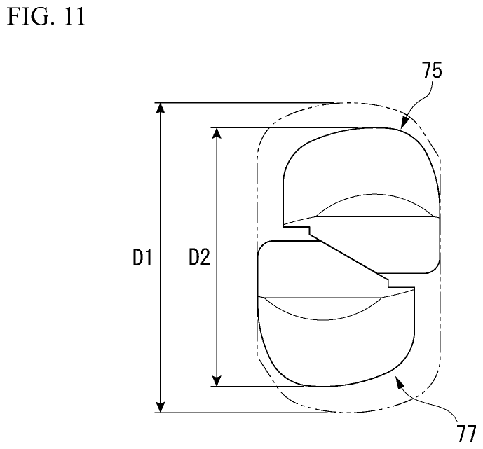

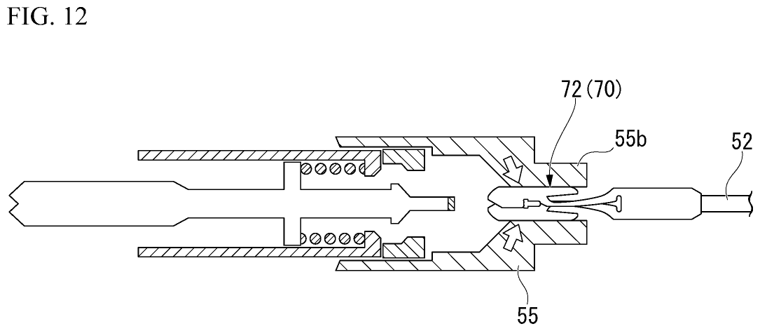

FIG. 11 shows a state in which the claws 75, 77 in the balanced state are viewed from the front side. In this state, the maximum radial dimension (maximum dimension in the direction orthogonal to the axis X1) D2 of the front portion 72 of the hook 70 is smaller than the maximum radial dimension D1 of the front portion 72 (shown by broken line in FIG. 7) in the state in which the hook 70 and the arm portion 20 are connected, and is smaller than the inner diameter of the small-diameter portion 55b. Therefore, the front portion 72 in the balanced state, in which the relative movement of the claw portions 75 and 77 has stopped, can pass through the small-diameter portion 55b as shown in FIG. 12.

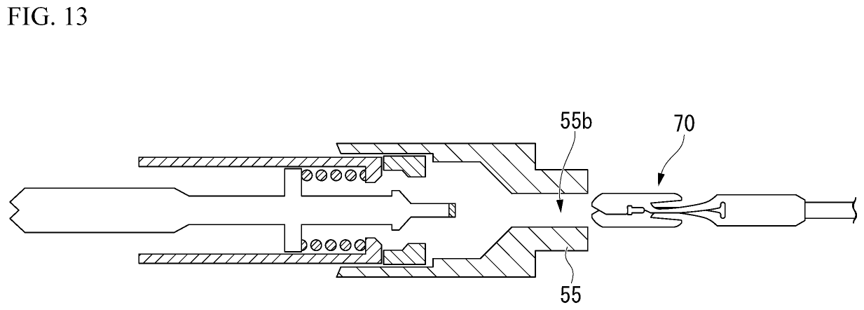

When the user further retracts the slider 62, the hook 70 passes through the small-diameter portion 55b while being deformed, and exits from the proximal end opening of the guide pipe 55 as shown in FIG. 13. When the hook 70 comes out of the guide pipe 55, the external force that opposes the traction of the operation wire 52 does not apply on the hook 70 such that the force required to retract the slider 62 is greatly reduced and the operation of the slider 62 becomes lighter. Therefore, the user can easily perceive that the clip unit 10 has been separated from the applicator 50.

When the user removes the endoscope and the applicator 50, a series of procedures is completed.

As described above, according to the ligation device 1 of the present embodiment, it is possible to prevent the connection between the clip unit 10 and the applicator 50 from being released before the arms of the clip unit 10 are locked in the case of ligating the rigid tissue due to the dimensional relationship between the regulating member 57 and the claws 75 and 77.

Further, simply by pressing the rear end portions of the claws 75, 77 against the reduced-diameter portion 55a of the guide pipe 55, the claws are opened and the connection between the clip unit 10 and the applicator 50 is released. Therefore, the user can release the connection by only continuously retracting the slider 62, and the force required at that time can be made smaller than the force required when locking the arm.

Further, the hook 70 after the connection is released can be pulled out from the guide pipe 55 through the small-diameter portion 55b of the guide pipe 55 by the claws 75, 77 being closer than the time of being connected. As a result, as described above, the operation of the slider 62 becomes significantly lighter, and it is possible to easily perceive that the clip unit 10 has been separated from the applicator 50.

By each of the above-mentioned features, in the ligation device 1, it is possible to achieve the both goals of reliably indwelling the clip unit 10 in the state of clamping the rigid tissue and making the operations to be easy to perform.

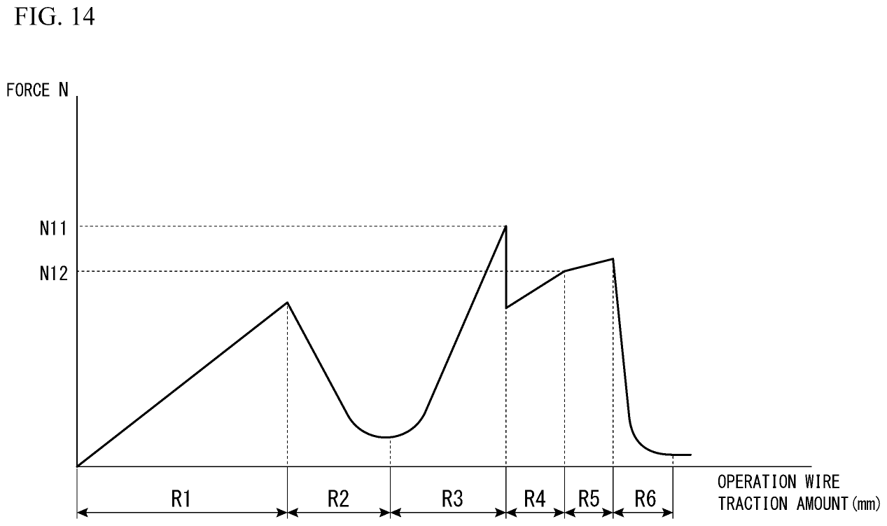

FIG. 14 shows the relationship between the traction amount of the operation wire and the generated force. When the operation wire is pulled by operating the slider 62 after the arm portion clamps the tissue, the force N is gradually increased by the reaction force received from the clamped tissue in the region R1 until almost the entire arm portion is pulled into the pressing tube.

Subsequently, in the region R2 until the proximal end portion of the arm portion reaches the proximal end opening of the pressing tube, the force N gradually decreases because the arm portion and the inner surface of the pressing tube are substantially parallel.

Subsequently, in the region R3 until the arm portion is locked, the force N increases again since the arm portion has to be deformed to make the locking portion 23 to get over the proximal end opening. Once the locking operation is finished, the force N drops once.

Subsequently, the force N increases again in the region R4 until the connection between the arm portion and the hook is released. Mechanically, if the maximum force value N2 in the region R4 is equal to or less than the maximum force value N1 in the region R3, the connection with the hook may be released before the arm portion is locked, and it is possible that the tissue cannot be ligated. However, in the present embodiment, due to the dimensional relationship between the regulation member 57 and the claws 75, 77, it is possible to physically prevent the connection from being released. Also, due to the operations of pressing the rear end portions of the claws 75, 77 to the reduced-diameter portion 55a of the guide pipe 55 so as to open the claw portion, it is possible to make the maximum force N2 to be equal to or less than the maximum force N1 and prevent the connection from being released before the arm portion is locked without relying on the rigidity of the claw portion.

Subsequently, the force N gradually increases in the region R5 until the claw portions 75, 77 enter the small-diameter portion 55b. In the region R6 after the claws 75, 77 have exited from the guide pipe 55, the force N decreases rapidly.

Although one embodiment of the present invention has been described above, the technical scope of the present invention is not limited to the above-described embodiment, and various changes or deletion may be made to each component within a range that does not deviate from the gist of the present invention.

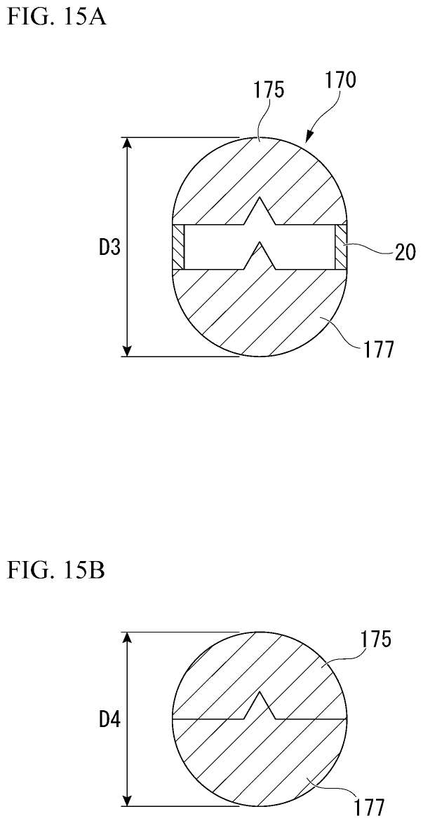

For example, when the claw portion passes through the small-diameter portion of the guide pipe, it does not have to be displaced in the left-right direction. In the modification examples shown in FIG. 15A and FIG. 15B, when the arm portion 20 does not exist between the claw portions 175, 177 due to the release of the connection, the claw portions 175, 177 approach each other and the maximum radial dimension D4 of the claw portion is smaller than the maximum radial dimension D3 at the time during the period when the connection is maintained. By setting the maximum radial dimension D4 to be equal to or less than the inner diameter of the small-diameter portion 55b, the hook after the connection is released can be made to enter the small-diameter portion and be pulled out from the guide pipe.

The cross-sectional area of the pair of claw portions in the longitudinal direction of the ligation device is preferably equal to or less than the cross-sectional area of the inner cavity (lumen) of the small-diameter portion. As a result, it is easy to make the small diameter portion to pass by only displacing the claw portion. If the cross-sectional area of the pair of claw portions after the displacement is the same as that of the inner cavity of the small-diameter portion, both are not deformed when the claw portions pass through the small-diameter portion, and the amount of operation force is unlikely to increase.

The pair of claw portions do not necessarily have to come into contact with each other when passing through the small-diameter portion.

In the present disclosure, the regulation member and the guide pipe are not essential. For example, a regulation portion may be provided in a part of the guide pipe, and the reduced-diameter portion or the small-diameter portion may be formed by molding the distal end portion of the sheath configuring the insertion portion.

The cross-sectional shape of the inner cavity of the small-diameter portion is not limited to a circular shape, and may be a polygon such as a square or the like. In this case, in the second state, if the claw portion is configured such that the maximum radial dimension of the approaching claw portion is equal to or less than the minimum radial dimension of the small-diameter portion, the claw portion can be approached and smoothly passed through the small-diameter portion. When the difference between the radial minimum dimension and the radial maximum dimension of the small-diameter part is small (for example, 0.1 mm or less), in the second state, if the claw portion is configured such that the maximum radial dimension of the approaching claw portion is equal to or less than the maximum radial dimension of the small-diameter portion, the same effect can be obtained by making the claw portion to pass through the small-diameter portion while at least one of the claw portion and the small-diameter portion is slightly deformed.

Although each preferred embodiment of the present invention has been described above together with each embodiment, the present invention is not limited to this embodiment and each embodiment. Configurations can be added, omitted, replaced, and other modifications without departing from the spirit of the present invention. Further, the present invention is not limited by the above description and is limited only by the appended claims.

What is claimed is:

1. A clip device, comprising:
a clip unit including an arm configured to open and close;
a hook configured to connect to and detach from the clip unit;
a wire configured to operate the clip unit and connected to the hook, a tubular member, the hook being inserted through the tubular member; and
a claw portion provided within the hook, wherein:
in a first state in which the hook is connected with the clip unit, the claw portion is configured to have a radial dimension larger than an inner diameter of the tubular member, and
in a second state in which the hook is disconnected from the clip unit, the radial dimension of the claw portion is equal to or less than the inner diameter of the tubular member.

2. The clip device according to claim 1, wherein when the hook is in the second state, the hook is deformed so that the radial dimension of the claw portion is equal to or less than the inner diameter of the tubular member.

3. The clip device according to claim 2, wherein when the hook is in the second state, the hook is deformed in a direction towards a longitudinal axis of the tubular member so that the radial dimension of the claw portion is equal to or less than the inner diameter of the tubular member.

4. The clip device according to claim 2, wherein:
the claw portion includes a plurality of claw portions are provided for clamping the clip unit, the plurality of claw portions being configured to detach from the clip unit and the hook, and
in the second state, the plurality of claw portions are deformed such that each claw portion is provided closer to each other than that in the first state so that the radial dimension of the claw portion is equal to or less than the inner diameter of the tubular member.

5. The clip device according to claim 1,
wherein the tubular member includes:
a reduced-diameter portion with an inner diameter that decreases along the tubular member towards a proximal end side, and
a small-diameter portion continuing to a proximal end side of the reduced-diameter portion and having an inner diameter that is equal to a smallest inner diameter of the reduced-diameter portion, and
the claw portion is configured to be inserted through the small-diameter portion when the hook is in the second state.

6. The clip device according to claim 5, wherein:
when the hook is in the first state, the radial dimension of the claw portion is larger than the inner diameter of the small-diameter portion, and
when the hook is in the second state, the radial dimension of the claw portion is equal to or less than the inner diameter of the small-diameter portion.

7. The clip device according to claim 5, wherein when the hook is in the first state, the claw portion is disposed at a distal end side of the small-diameter portion.

8. The clip device according to claim 1, wherein:
when the hook is detachably connected to the clip unit, the hook is deformed in a first direction along a radial direction of the tubular member, and
the claw portion is configured to displace in a second direction intersecting the first direction and an axial direction of the tubular member.

9. The clip device according to claim 8, wherein:
the claw portion has an inclined surface that inclines relative to the second direction, and
when the hook is in the second state, the radial dimension of the claw portion is equal to or less than the inner diameter of a small-diameter portion as the claw portion moves along the inclined surface.

10. A clip device, comprising:
a clip unit including an arm;
a hook detachably connected to the clip unit;
a wire configured to operate the clip unit and connected to the hook; and
a pair of claws provided in the hook, wherein:
the pair of claws are disposed at positions sandwiching the clip unit in a first direction orthogonal to a longitudinal direction of the hook, and
the pair of claws are disposed at asymmetrical positions in a second direction orthogonal to the first direction and the longitudinal direction.

11. The clip device according to claim 10, the pair of claws including a first claw and a second claw, the first claw including a first surface configured to engage with the clip unit and a second surface being inclined in a direction away from the second claw.

12. The clip device according to claim 11, wherein:
the second claw includes a first surface and a second surface, the first surface of the second claw configured to engage with the clip unit and the second surface is inclined in a direction away from the first claw,
when the hook is in a first state in which the hook is connected to the clip unit, the pair of claws are disposed at a first position wherein the second surface of the first claw is separate from the second surface of the second claw, and
when the hook is in a second state in which the hook is disconnected to the clip unit, the second surface of the first claw contacts the second surface of the second claw.

13. The clip device according to claim 12, wherein the second surface of the first claw and the second surface of the second claw are parallel to each other.

14. The clip device according to claim 12, wherein the pair of claws are configured to displace in the second direction when the pair of claws transition from the first position to the second position.

15. The clip device according to claim 12, wherein a dimension of the pair of claws in the first direction when the pair of claws are disposed at the second position is smaller than the dimension of the pair of claws when the pair of claws are disposed at the first position.

16. A tissue closure method, comprising:
pulling a wire to grasp a tissue by a clip detachably connected to the wire;
causing a link member to contact an inner circumferential surface of a tubular member by pulling the wire in a state in which the tissue is grasped by the clip, the link member being configured to detachably connect the clip and the wire, and the wire being insertable through the tubular member;
releasing a connection between the link member and the clip by pulling the wire when the link member is in contact with the inner circumferential surface of the tubular member; and
separating the link member from the tubular member by pulling the wire when the connection between the link member and the clip is released.

17. The tissue closure method according to claim 16,
engaging and sandwiching the clip with a hook that includes a plurality of claw portions, and
when the link member is in contact with the inner circumferential surface of the tubular member, and the wire is pulled, the connection between the link member and the clip is released by causing the plurality of claw portions to separate from each other.

18. The tissue closure method according to claim 17, wherein when the wire is pulled in the state in which the connection between the link member and the clip is released, the link member is deformed to separate from the tubular member by moving the plurality of claw portions in a direction so as to make the plurality of claw portions approach each other.

19. The tissue closure method according to claim 18,
wherein the tubular member includes a small-diameter portion and a large-diameter portion having a larger inner diameter than that of the small-diameter portion, the large-diameter portion being disposed to be closer to the clip, and
when the wire is pulled in the state in which the connection between the link member and the clip is released, the link member is deformed by moving the link member from a position on the large-diameter portion toward a position on the small-diameter portion.

20. The tissue closure method according to claim 19, further comprising:
pulling the wire for deforming the link member in a second direction intersecting with both a first direction in which the plurality of claw portions sandwiches the clip and an axial direction of the tubular member when the link member is released from the clip.
